(19) United States
(12) Patent Application Publication
(10) Pub. No.: US 2012/0226220 A1
(43) Pub. Date: Sep. 6, 2012

(54) SHIELDABLE UNIT DOSE MEDICAL NEEDLE ASSEMBLIES

(75) Inventors: Alfred W. Prais, Hewitt, NJ (US); Richard James Caizza, Verona, NJ (US); Gary Henniger, Wayne, NJ (US); Espen D. Karteraas, Tuxedo, NY (US)

(73) Assignee: BECTON, DICKINSON AND COMPANY, Franklin Lakes, NJ (US)

(21) Appl. No.: 13/471,989

(22) Filed: May 15, 2012

Related U.S. Application Data
(63) Continuation of application No. 11/384,759, filed on Mar. 20, 2006, now abandoned.
Application No. 11/384,759 is a continuation of application No. 10/402,904, filed on Mar. 28, 2003, now abandoned.
Application No. 10/402,904 is a continuation-in-part of application No. 10/141,538, filed on May 9, 2002, now abandoned.
(60) Provisional application No. 60/344,304, filed on Dec. 28, 2001.

(51) Int. Cl. A61M 5/32; A61M 37/00

(52) U.S. Cl. 604/22; 604/198; 604/506

(57) ABSTRACT

A shieldable unit dose assembly includes a needle holding member, a safety shield member disposed around the needle holding member, and a unit dose needle including a hub which is connected to the needle holding member. The needle holder member includes a male tapering surface and the hub of the unit dose needle includes a female tapering surface for connection therebetween. The unit dose needle includes a solid elongated unit dose needle having a patient end for containing and administering a unit dose of a vaccine. The telescoping safety shield member is disposed around the needle holding member, and is axially movable from a non-shielding position surrounding at least a portion of the needle holding member to a telescoped shielding position encompassing the patient end of the unit dose needle. The safety shield member may be locked into the shielding position.

### CROSS REFERENCE TO RELATED APPLICATIONS

This application is a continuation of U.S. patent application Ser. No. 11/384,759, filed Mar. 20, 2006, entitled Shieldable Unit Dose Medical Needle Assemblies“, which is a continuation of U.S. patent application Ser. No. 10/402,904, filed Mar. 28, 2003, entitled “Shieldable Unit Dose Medical Needle Assemblies”, which is a continuation-in-part of U.S. patent application Ser. No. 10/141,538, filed May 9, 2002, entitled “Medical Needle Assemblies”, which claims priority to U.S. Provisional Patent Application Ser. No. 60/344,304, filed Dec. 28, 2001, entitled “Bifurcated Needle Assembly”, the contents of all of which are herein incorporated by reference.

### BACKGROUND OF THE INVENTION

1. Field of the Invention

The present invention relates to needles for use in medical procedures and, in particular, to safety shielded needles and needle assemblies used in medical procedures.

2. Description of Related Art

Bifurcated or forked end needles are well-known for providing a simple and effective means for a doctor to administer a vaccine. During use, the bifurcated tip of the bifurcated needle is put into contact with either a dried or liquid substance, which adheres to the bifurcated needle tip. The bifurcated needle tip is then put into contact with the skin of the patient who is being administered the vaccination. The skin is either scratched or pierced with the needle tip so that the vaccination material may be absorbed into the skin of the patient. An alternative method of delivering the vaccination includes placing a drop of the vaccine onto the skin of the patient and contacting the skin of the patient with the bifurcated needle tip through the drop of vaccination. Alternatively, a standard pointed needle tip may also be used when the drop of vaccination is applied directly to the skin of the patient.

The bifurcated needle is considered a significant medical advancement because it has allowed more people to be vaccinated with less serum. This has been especially important for those living in less developed areas because of the efficient and easy to use design, as well as the ease of replication.

Vaccination effectiveness, however, is reduced if the bifurcated needle is reused too many times. Moreover, reuse of such vaccination needles exposes patients to the risk of transmission of infectious diseases through percutaneous contact through the skin. Additionally, medical care workers using traditional vaccination needles are at an increased risk of exposure to infectious diseases due to the design of such needles, which makes them difficult to handle, as well as due to the repeated use of such needles.

In particular, bifurcated needles used to administer vaccinations are not traditionally sterilized or packaged in a single-use container that would enable convenient storage and subsequent use. Additionally, such needles have traditionally been difficult to handle in that they typically do not include a hub attached to the opposite end of a needle from the tip, and do not typically include any sort of shield for protection from the needle prior to and during use.

For example, U.S. Pat. No. 3,194,237 to Rubin discloses a vaccinating needle having a main shank with a pair of prongs at one end that define a slot of predetermined length, width and depth therebetween to hold an amount of liquid by capillary action. The shank of the needle is of sufficient length so that the non-prong end will function as a handle. U.S. Pat. No. 3,948,261 to Steiner discloses a reusable unit dose container for vaccines contained within a rigid receptacle, with a compressible closure for supporting a bifurcated needle bearing dried vaccine. The closure is adapted to support the needle in the container during a lyophilizing process while liquid vaccine is dried on the needle. The closure has grooves which permit the vaporized liquid from the vaccine to be withdrawn from the receptacle during lyophilizing, and can further seal the container.

While shieldable syringes or needle assemblies are well known in the art for needles used to inject fluids and medicine into the circulatory system of the patient (i.e., veni-puncture), such shielding has not previously been used in connection with vaccination needles such as bifurcated needles. Thus, a need exists for a safety assembly for use with a unit dose vaccination needle that is easily manufactured, simple to use, and may be shielded to prevent accidental needle stick wounds.

### SUMMARY OF THE INVENTION

The present invention is directed to a shieldable unit dose assembly for administering a unit dose of vaccine. The assembly generally includes a needle holding member having a proximal end and a distal end, with the distal end including a male tapering surface. The assembly further includes a unit dose needle assembly supported at the distal end of the needle holding member. The unit dose needle assembly includes a solid elongated unit dose needle having a patient end for containing and administering a unit dose of a vaccine and a hub supporting the unit dose needle. Desirably, the unit dose needle is a bifurcated needle having two pointed prongs capable of penetrating or abrading the skin of the patient, with the prongs being separated by a U-shaped channel capable of holding a unit dose of vaccine. The hub includes a female tapering surface in engagement with the male tapering surface at the distal end of the needle holding member. The assembly further includes a telescoping safety shield member disposed around the needle holding member and axially movable from a non-shielding position surrounding at least a portion of the needle holding member to a telescoped shielding position encompassing the patient end of the unit dose needle.

The needle holding member may include a locking member at a distal end thereof, and the safety shield member may include an internal locking recess at a proximal end thereof adapted to cooperate with the locking member when the safety shield member is in the shielding position to prevent the safety shield member from returning to the non-shielding position. The locking member may comprise a locking collar having an exterior wedge for engaging the internal locking recess to prevent the safety shield member from returning to the non-shielding position. Further, the assembly may include means for indicating when the safety shield member is in the fully shielding position, such as a color indicator.

In a further embodiment, the present invention is directed to a shieldable needle assembly including a unit dose needle assembly connected to a needle holder assembly. The unit dose needle assembly includes a hub having a female tapering surface at a proximal end thereof and a solid elongated unit dose needle extending from a distal end thereof. The unit dose needle is of a length which is capable of retrieving a unit dose of a vaccine from a separate container, and includes a patient end for containing and administering the unit dose of a vaccine, such as a bifurcated pronged end with a U-shaped channel The needle holding assembly includes an elongated body extending between a proximal end and a distal end. The distal end includes a male tapering surface in engagement with the female tapering surface of the hub of the unit dose needle assembly, and also includes an annular collar having internal threads in threaded engagement with corresponding structure on the proximal end of the hub. The needle holding assembly further includes a telescoping safety shield member disposed concentrically around the elongated body with at least a portion of the proximal end of the body extending from a proximal end of the safety shield member. The safety shield member is axially movable from a non-shielding position in which the unit dose needle is exposed to a telescoped shielding position encompassing the length of the unit dose needle including the patient end.

In a further embodiment, the present invention is directed to a method for administering a vaccine. In the method, a shieldable medical needle assembly comprising a unit dose needle assembly attached to a needle holding assembly is provided. The unit dose needle assembly includes a solid elongated unit dose needle including a bifurcated pronged patient end for containing a unit dose of a vaccine. The needle holding assembly includes an elongated body with a proximal end and a distal end, and further includes a telescoping safety shield member disposed concentrically around the elongated body with at least a portion of the proximal end of the body extending from the safety shield member. The bifurcated pronged patient end of the unit dose needle is first contacted with a vaccine so as to contain at least a portion of the vaccine on the bifurcated pronged patient end. The bifurcated pronged patient end of the unit dose needle is thereafter contacted with a patient's skin so as to cause the bifurcated pronged patient end to abrade the patient's skin. The safety shield member is thereafter axially moved in a direction toward the bifurcated pronged patient end. As such, the shield member is moved from a non-shielding position in which the safety shield member is disposed concentrically around the elongated body and the unit dose needle is exposed to a telescoped shielding position encompassing the length of the unit dose needle including the bifurcated pronged patient end.

Further details and advantages of the present invention will become apparent from the following detailed description, when read in conjunction with the drawings.

### BRIEF DESCRIPTION OF THE DRAWINGS

FIG. 1 is a perspective view of a shieldable needle assembly of the present invention;

FIG. 2 is an exploded view of the shieldable needle assembly shown in FIG. 1;

FIG. 3 is an exploded perspective view of the unit dose needle assembly of FIG. 1;

FIG. 4 is a side cross-sectional view of the unit dose needle assembly of FIG. 3;

FIG. 5 is a side cross-sectional view of the shieldable needle assembly of FIG. 1;

FIG. 6 is an enlarged partial cross-sectional view of a distal end of the needle holding member;

FIG. 7 is an enlarged plan view of a locking member adapted for connection to the needle holding member of the present invention;

FIG. 8 is a cross-sectional view of the locking member of FIG. 7;

FIG. 9 is an enlarged partial cross-sectional view of the distal end of the needle holding assembly positioned within a safety shield;

FIG. 10 is a perspective view of the shieldable needle assembly showing the safety shield member in a telescoped, shielding position;

FIG. 11 is a side cross-sectional view showing the connection between the safety shield member, the locking member, and the needle holding member, with the safety shield member in the shielding position as shown in FIG. 10; and

FIG. 12 is a perspective view of a shieldable needle assembly in an alternate embodiment including color indicator means.

### DESCRIPTION OF THE PREFERRED EMBODIMENTS

In the following description and accompanying drawings, like reference numbers, as used in the various figures, refer to like features or elements. The terms top and bottom, as used herein, refer to the orientation of a given element as shown in the drawings.

For purposes of the description hereinafter, the terms “upper”, “lower”, “right”, “left”, “vertical”, “horizontal”, “top”, “bottom”, and derivatives thereof shall relate to the invention, as it is oriented in the drawing figures. However, it is to be understood that the invention may assume various alternative variations and step sequences except where expressly specified to the contrary. It is also to be understood that the specific devices and processes illustrated in the attached drawings and described in the following text are simply exemplary embodiments of the invention. Hence, specific dimensions and other physical characteristics related to the embodiments disclosed hereinafter are not to be considered limiting.

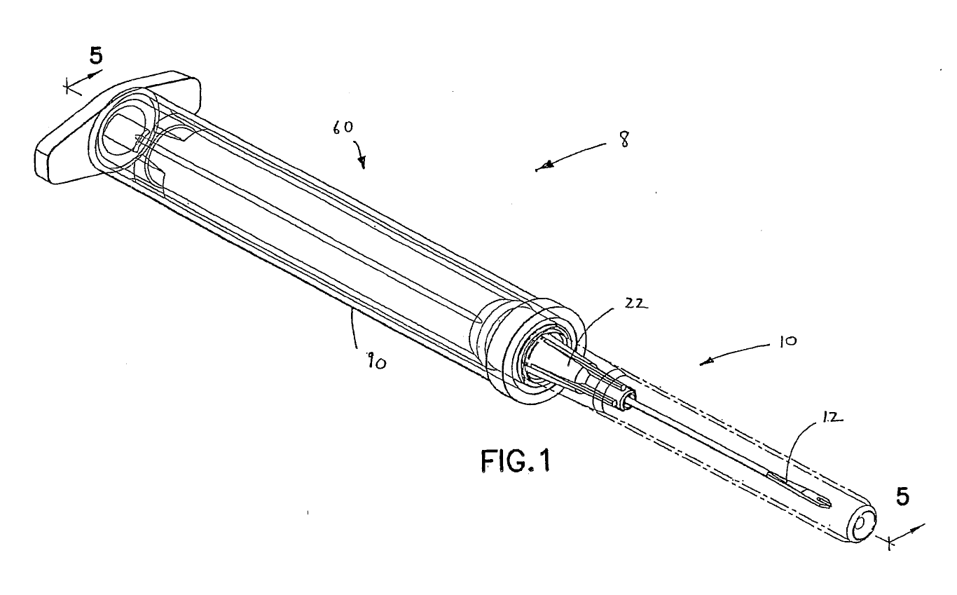

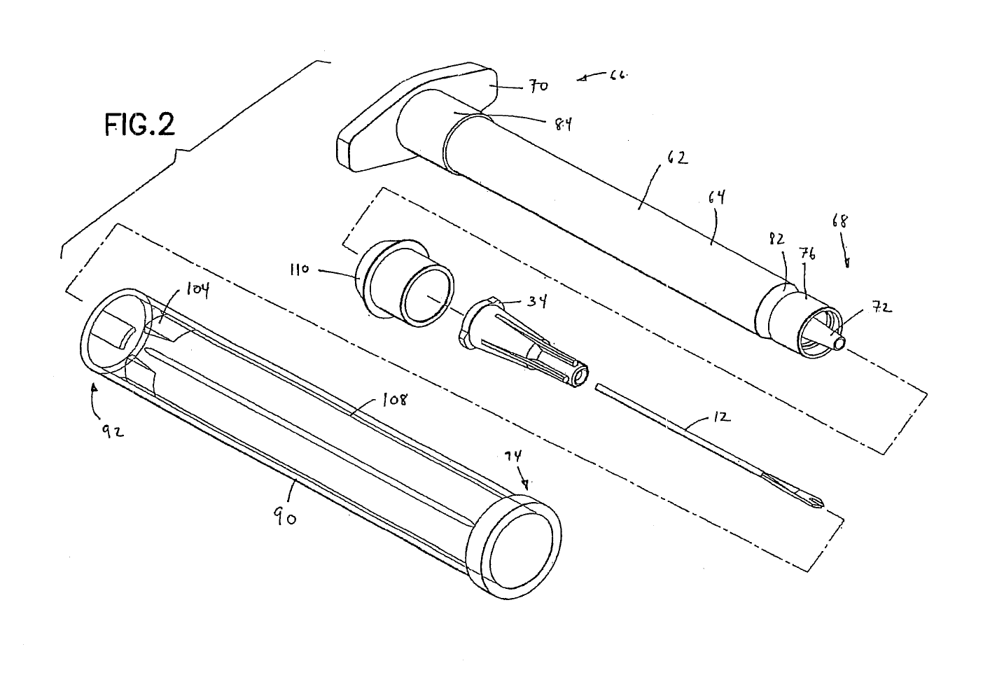

Referring to the drawings in which like reference characters refer to like parts throughout the several views thereof, FIGS. 1-2 depict a shieldable medical needle assembly 8 in accordance with the present invention and the related features. The shieldable assembly 8 includes a unit dose needle assembly 10 connected to a needle holding assembly 60. The shieldable assembly 8 is intended for use for the administration of vaccines applied to or through the skin of the patient, and is intended as a single-use vaccination needle assembly including features to provide ease of use for the medical practitioner and safety shielding of the needle after use, as will be described in more detail herein.

Generally, needle holding assembly 60 includes needle holding member 62 which is defined by a generally cylindrical body 64 extending between proximal end 66 and distal end 68. The cylindrical body 64 may be a hollow member, such as a conventional syringe barrel, or may be a solid member. As will be discussed further herein, proximal end 66 of the body 64 of needle holding member 62 desirably includes structure for grasping needle holding member 62, such as a circumferential flange or a pair of flange tabs 70.

The needle holding member 62 includes a tapered tip 72 projecting distally therefrom at distal end 68. Tapered tip 72 includes a male tapering surface such as a male luer taper 74. It is noted, however, that tapered tip 72 need not have any opening therethrough, and may be a solid member, which may provide additional structural integrity to the tapered tip 72. Needle holding member 62 may also include a luer collar 76 at distal end 68, which is generally adjacent tapered tip 72 and generally surrounds tapered tip 72. Luer collar 76 may include a plurality of internal threads 78 for threadably receiving a hub of a unit dose needle assembly, as explained further herein. The luer collar 76 defines an external groove 80 adjacent an annular shoulder 82 defined by body 64 of needle holding member 62.

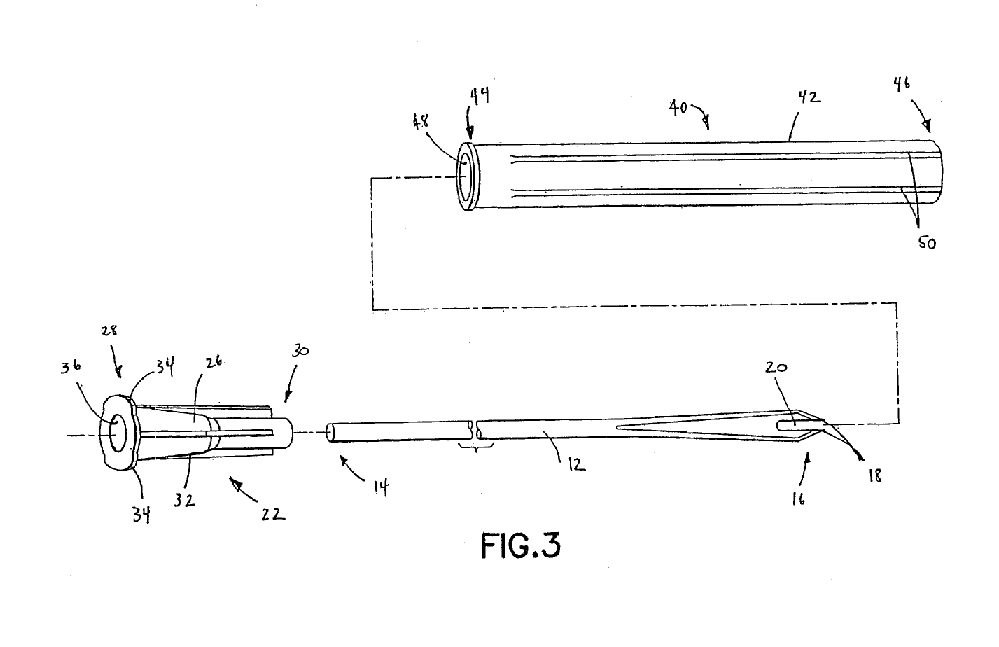

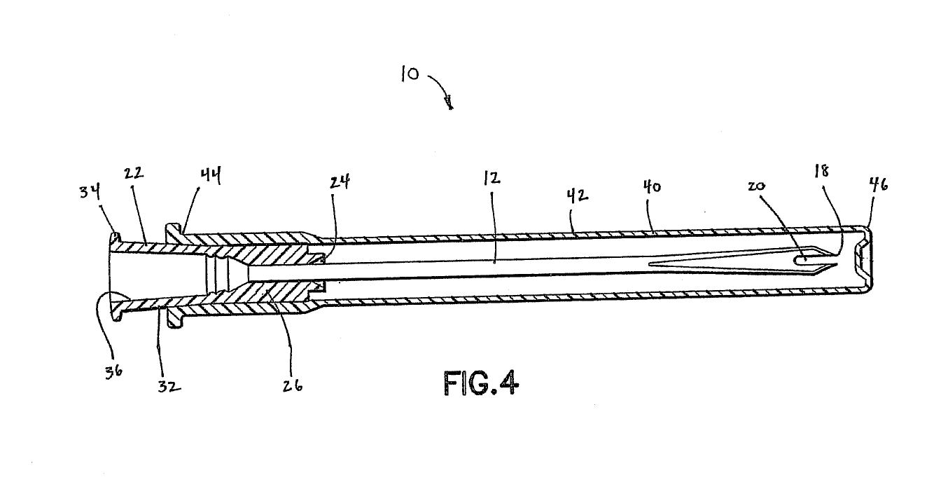

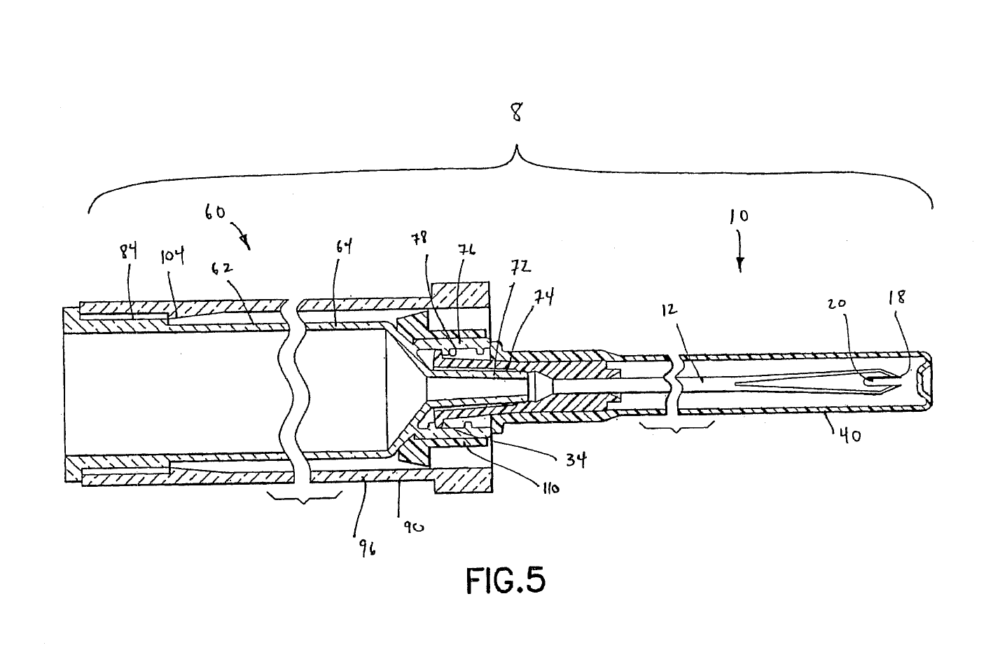

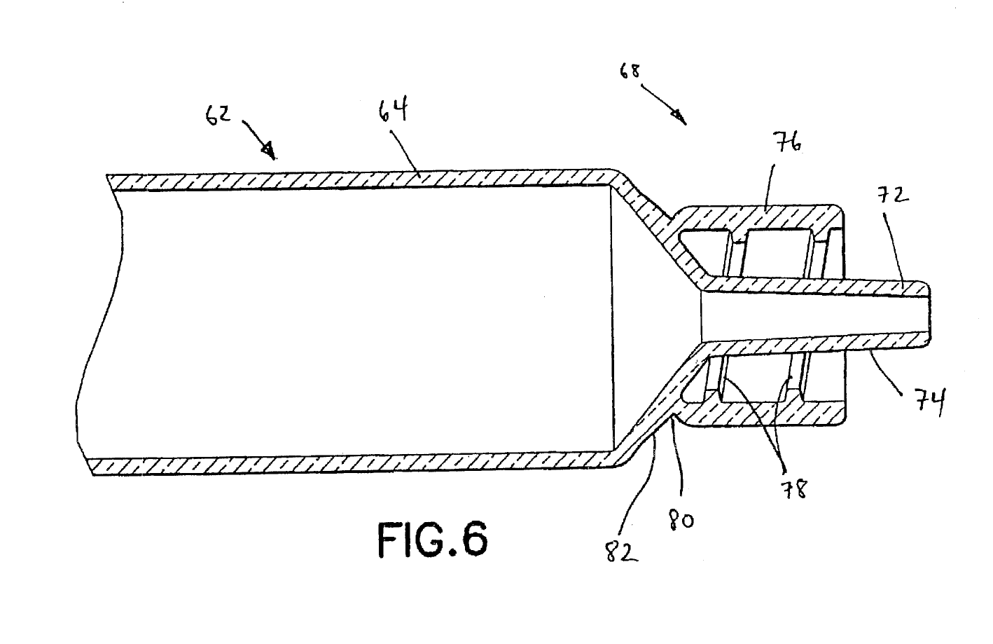

Referring generally to FIGS. 3-4, the shieldable needle assembly 8 further comprises a unit dose needle assembly 10 supported by the needle holding assembly 60 at the distal end 68 of needle holding member 62. The unit dose needle assembly 10 generally includes a unit dose needle for administering a unit dose of a vaccine. Desirably, the unit dose needle is a bifurcated needle, such as bifurcated needle 12, which is supported by a hub 22, and enclosed within a needle shield 40. While unit dose needle assembly 10 is described herein in terms of a preferred embodiment including bifurcated needle 12, unit dose needle assembly 10 may include any unit dose needle capable of administering a unit dose of a vaccine, such as in a dry powder or liquid form, as is well known in the art. Desirably, the unit dose needle is sterilized and maintained in a sterile condition until used, and may be safely disposed of along with the shieldable medical assembly 8 at the conclusion of a vaccination procedure, for example.

Bifurcated needle 12 is a solid, elongated needle structure extending between a non-patient end at proximal end 14, and an opposed prong end or patient end at distal end 16. Bifurcated needle 12 is provided with two sharp prongs 18 positioned at a distal end 16 of the needle. The prongs 18 are separated by a U-shaped channel 20 configured to hold a unit dose of vaccine. The prongs 18 are intended to penetrate or abrade the skin of the patient to administer the vaccine disposed in the U-shaped channel 20. Bifurcated needle 12 may be constructed of any material known in the art, such as metal or plastic, and is desirably constructed of a medical grade surgical steel.

Hub 22 is fixedly attached to the proximal end 14 of bifurcated needle 12, such as through an adhesive joint 24. Adhesive joint 24 may be provided through any adhesive capable of fixedly attaching or adhering bifurcated needle 12 to hub 22, such as an epoxy or equivalent adhesive. Hub 22 includes a hub housing 26 including a proximal end 28 and a distal end 30, with at least a portion of the external surface of hub housing 26 defining an outer tapered surface 32 extending therealong. Distal end 30 of hub 22 may include an internal bore having an internal diameter of approximately the same size as the outer diameter of the proximal end 14 of bifurcated needle 12, for accommodating and fixedly adhering bifurcated needle 12 within such an internal bore of hub 22.

The hub 22 further includes means for attaching the unit dose needle assembly 10 to the needle holding assembly 60. Desirably, the hub 22 includes luer lugs 34 extending about the proximal end 28 thereof, with an internal luer taper 36 which extends internally within a portion of hub housing 26. Internal luer taper 36 is a female tapering surface which extends internally within at least a portion of hub housing 26, for engagement with the male luer taper 74 of needle holding member 62. In this manner, unit dose needle assembly 10 can be attached to needle holding assembly 60, which can provide shieldable medical assembly 8 with an appropriate handle portion for the assembly, thereby facilitating ease of use of the assembly. While not shown, internal luer taper 36 may include internal threads for establishing threaded engagement with a corresponding threaded surface of the needle holding member 62.

While the present invention is described in terms of a unit dose needle assembly 10 separately attached to a needle holding assembly 60, it is contemplated that the unit dose needle assembly 10 and the needle holding assembly 60 can be permanently attached or integrally formed, thereby providing shieldable medical assembly 8 as a single structure.

Unit dose needle assembly 10 may further include a needle shield 40 extending about bifurcated needle 12. Needle shield 40 is of a generally tubular hollow construction, including tubular shield housing 42 extending between a proximal end 44 and a distal end 46, with the tubular shape of shield housing 42 forming an internal opening 48 extending through needle shield 40. Proximal end 44 of needle shield 40 is generally open-ended, forming a passage for access to internal opening 48, while distal end 46 is closed-ended, forming a wall. Needle shield 40 extends about bifurcated needle 12, thereby containing bifurcated needle 12 within internal opening 48. Proximal end 44 may further be provided with a lip extending circumferentially about the open ended passage. Proximal end 44 removably engages the hub 22 along the tapered surface 32 to form an air-tight seal, completely concealing the bifurcated needle 12 and associated prongs 18 therein in a sterile, air-tight manner.

The needle shield 40 serves to protect the bifurcated needle 12 from damage and exposure to soils or other contaminants during shipping and storage, and prior to use of the unit dose needle assembly. The needle shield 40 also provides protection to personnel from needle sticks prior to removing the needle shield 40 for use.

The hub 22 and needle shield 40 may be constructed of any material, and are desirably constructed of a moldable plastic material. Suitable moldable plastics include, but are not limited to polyethylenes, polypropylenes, polyamides, polyesters and fluorinated polyethylenes. Preferably, hub 22 is constructed of a rigid material, while needle shield 40 may be formed from a non-rigid material. By non-rigid material, what is meant is a material that is sufficiently flexible to conform to the tapered surface 32 of the hub 22 to form an air-tight seal. While needle shield 40 may be desirably formed from polypropylene, any material known by those of skill in the art to facilitate sterilization of the unit dose needle 10 may also be used. Needle shield 40 may further include external ribs 50 integrally molded with shield housing 42 and extending longitudinally along the outer surface of shield housing 42 between proximal end 44 and distal end 46. Such external ribs 50 provide further structural integrity to needle shield 40, which is particularly useful during packaging and storage.

The unit dose needle, i.e., bifurcated needle 12, may be used to administer vaccine that is in any suitable foam. Suitable physical forms for the vaccine include, but are not limited to liquids, such as solutions, emulsions, and dispersions, or dry powders. Typically, the vaccine will be in a liquid form. Moreover, the vaccine may be associated with unit dose needle assembly 10 during storage, and may therefore be contained within the U-shaped channel 20 prior to removal of needle shield 40. Alternatively, the vaccine may be provided as a separate component in a separate container, with bifurcated needle 12 being contacted with the vaccine after removing needle shield 40 therefrom just prior to use of the needle for administration of the vaccine. In this manner, it is important that the unit dose needle be of sufficient length so as to be able to contact the vaccine within its storage container. For example, vaccines are typically stored in closed end containers or vials. The unit dose needle must be of a sufficient length so as to be able to extend into the container or vial and contact the vaccine therein. Typically, the bifurcated needle 12 will have a length of about 1.8 inches, and the unit dose needle including the hub 22 will have an overall length of about 2.2 inches.

As noted, needle shield 40 sealingly mates with hub 22 to provide an air-tight connection therebetween, with bifurcated needle 12 contained within the air-tight environment in internal opening 48 of needle shield 40. Such an air-tight arrangement provides unit dose needle assembly 10 as a self-contained assembly, in the form of a complete, shielded, sterile, single-use unit dose needle assembly, which can be shipped in this form. Alternatively, this unit dose needle assembly 10 may be further packaged to provide additional sterility to the assembly.

As noted, unit dose needle assembly 10 is connected with needle holding assembly 60. Needle holding assembly 60 further includes a telescoping safety shield member 90 disposed around the body 64 of needle holding member 62. The safety shield member 90 includes a proximal end 92, a distal end 94, and a rigid tubular sidewall 96 extending therebetween. The overall length of safety shield member 90 is slightly less than the body 64 of needle holding member 62, thereby providing a portion of needle holding member 62 to grasp for relative axial movement of the safety shield member 90 and the needle holding member 62. The overall length of safety shield member 90 is also sufficient so as to completely encompass the unit dose needle, i.e. bifurcated needle 12 and prongs 18.

Interior portions of the safety shield member 90 adjacent the proximal end 92 include an inward directed locking shoulder 98 having a proximal face 100 and a radially aligned distal locking face 102. A locking recess 103 is defined distally forward of the locking shoulder 98. The locking recess 103 is formed by a locking face of the locking shoulder 98 and a locking pawl 104 located distally forward of the locking shoulder 98. The locking pawl 104 includes a locking pawl face 106, which further defines the locking recess 103. The interior surface of safety shield member 90 may further include longitudinal ribs extending therealong, which facilitate axial movement of safety shield member 90 during use, as will be explained in further detail herein.

Needle holding assembly 60 further includes a locking member in the form of locking collar 110 disposed around needle holding member 62 at distal end 68 thereof. The locking collar 110 includes a proximal end 114, a distal end 116, and a generally tubular sidewall 118 extending therebetween. The locking collar 110 also includes a portion cooperating with the external groove 80. The locking collar 110 is adapted to connect and lock the telescoping safety shield member 90 to the needle holding member 62.

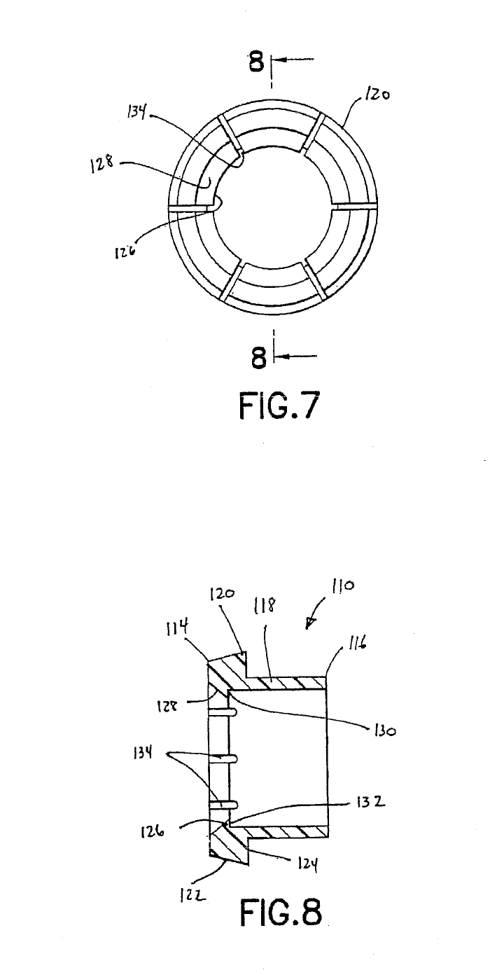

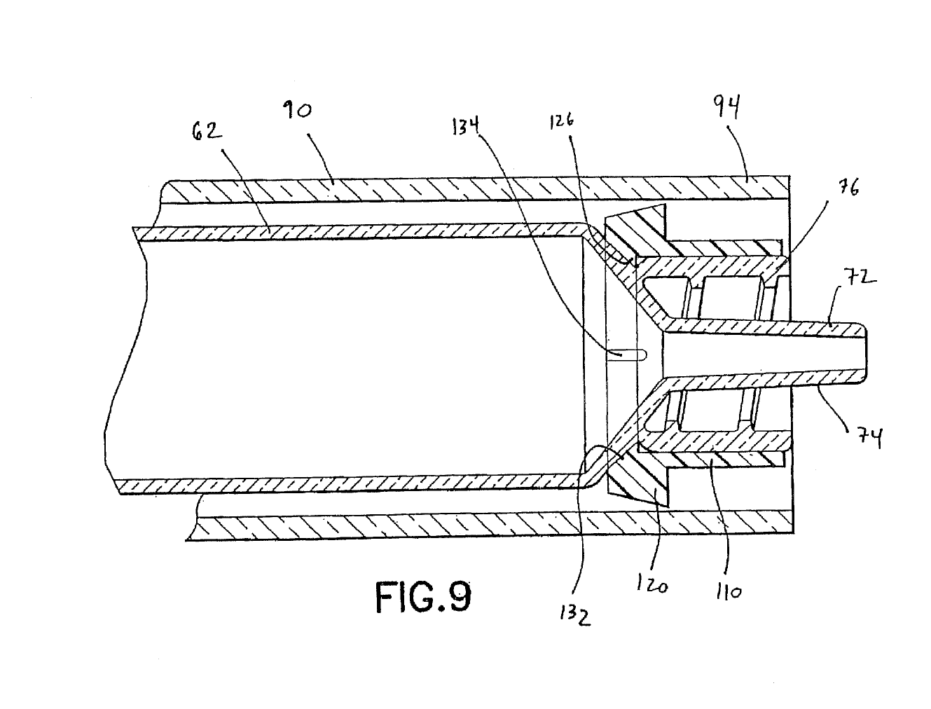

More particularly, the locking collar 110 includes an exterior wedge 120 having a frustom-shaped proximal face 122 and a radially aligned distal face 124. The locking collar 110 further includes an interior wedge 126 having an outward tapered proximal face 128 and a frustom-shaped distal face 130. The proximal and distal faces of the interior wedge 126 foam a barbed portion 132 adapted to cooperate with the external groove 80 defined in the body 64 of needle holding member 62. As shown in FIGS. 7-8, the locking collar 110 is further defined by a plurality of slots 134 extending distally from the proximal end 114. The slots 134 permit the locking collar 110 to tightly engage the external groove 80 to secure the connection between the safety shield member 90 and the needle holding member 62 as discussed hereinafter.

The needle holding member 62, shield member 90, and the locking collar 110 may be made of any of the materials identified previously in connection with the unit dose needle assembly 10 and, more particularly, the hub 22 of the unit dose needle assembly 10. Desirably, the locking collar 110 is made of a sufficiently resiliently deformable material such that it may be positioned over the distal end 68 of the body 64 and resiliently engage the external groove 80 defined in the needle holding member 62.

The shieldable medical assembly 8 may be assembled by initially mounting the safety shield member 90 over the needle holding member 62. The outside diameter of body 64 of the needle holding member 62 adjacent proximal end 66 may be formed slightly larger than the outside diameter of the remainder of body 64 forming an enlarged portion 84, so that the proximal end 66 of the needle holding member 62 may frictionally and releasably engage locking shoulder 98 for holding the safety shield member 90 in the proximal or non-shielding position on the needle holding member 62. The locking collar 110 may then be advanced on to the distal end 68 of the needle holding member 62, with the slots 134 defined in the locking collar 110 permitting the locking collar 110 to expand and fit onto the tip 72. In particular, the locking collar 110 engages the luer collar 76 located at the distal end 68 of the needle holding member 62. The interior wedge 126 of the locking collar 110 engages the external groove 80 defined in the luer collar 76. The tapered face 128 of the locking collar 110 aids in the expansion of the locking collar 110 as it is positioned over the luer collar 76. Once the locking collar 110 engages the external groove 80, the locking collar 110 returns to an undeflected condition, with the interior wedge 126 securely engaged in the external groove 80.

Once the safety shield member 90 is positioned about the needle holding member 62 and locking collar 110, the unit dose needle assembly 10 may be connected to the needle holding member 62. In particular, the hub 22 of the unit dose needle assembly 10 is threadably engaged with internal threads 78 defined in the luer collar 76, with female internal luer taper 36 frictionally engaging male external luer taper 74. The shieldable medical assembly 8 is now ready for use in a vaccination procedure, as an example.

The unit dose needle may be pre-supplied with a vaccine liquid. In which case, once the needle shield is removed, the unit dose needle is ready to be used in the vaccination procedure. Alternatively, the needle shield may be removed and the unit dose needle may be contacted with a vaccine. Once used in a vaccination procedure, the unit dose needle is considered contaminated. To prevent accidental needle sticks with the used unit dose needle and to prevent unauthorized use of the shieldable medical assembly, the technician using the assembly urges the safety shield member 90 distally forward relative to the needle holding member by holding flange tabs 70 and forcing the safety shield member 90 toward the distal end 68. Initial distal forces on the safety shield member 90 will disengage the locking shoulder 98 from its frictional engagement with the slightly enlarged portion 84 at the proximal end 66 of the needle holding member 62. Further distal movement will proceed without substantial interference.

Moreover, interior ribs 108 on the interior surface of tubular wall 96 of safety shield member 90 assist in proper axial movement of safety shield member 90 with respect to needle holding member 62. In particular, interior ribs 108 glide along an outside edge of exterior wedge 120 of locking collar 110 in a frictional manner during such movement from the non-shielding position to the shielding position. Such frictional gliding prevents safety shield assembly 90 from being easily rotated about needle holding member 62, and therefore assists in maintaining axial movement thereof.

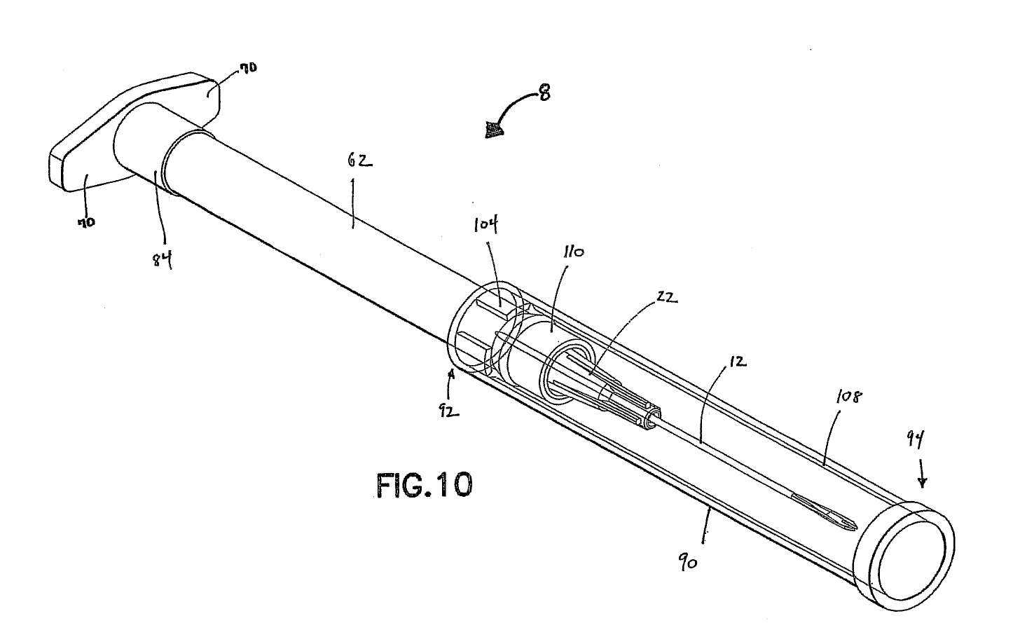

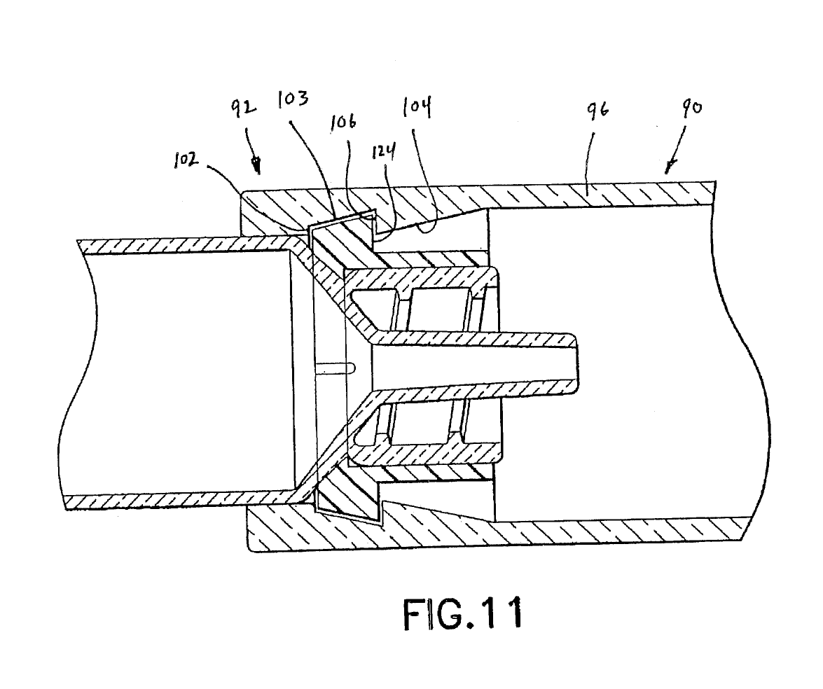

As the safety shield member 90 is telescoped over the unit dose needle toward a shielding position as shown in FIG. 10, the proximal pawl face 106 of the locking pawl 104 engages the exterior wedge 120 on the locking collar 110. This initial engagement will generate a slight radial expansion of the safety shield member 90 near its proximal end 92 to prevent further distal movement of the locking shoulder member 98. After further distal movement of the locking shield member 90, the exterior wedge 120 is received in a locking recess 103 between the distal locking face 102 and the pawl face 106, and the safety shield member resiliently returns to an unexpanded condition. The engagement of the exterior wedge 120 in the locking recess 103 prevents distal or proximal movement of the safety shield member 90. Additionally, the engagement of the exterior wedge 120 and the locking recess 103 simultaneously tightens the interior wedge 126 into the external groove 80 in the luer collar 76. Further, additional distal movement is also prevented by engagement of the locking shoulder 98 with the proximal face 122 of the exterior wedge 120. In summary, using normal fingertip force, movement of the safety shield member 90 is prevented by the interior and exterior wedges 126, 120, respectively, on the locking collar 110, which mate with the external groove 80 in the luer collar 76 and the locking recess 103 in the safety shield member 90. The above described wedging interaction between the safety shield member 90 and locking collar 110 are typically sufficient to prevent tampering with the safety shield member 90 once extended to its shielding position.

As indicated previously, a return or proximal movement of the safety shield member 90 to the non-shielding position with the unit dose needle exposed is prevented by the interaction between the proximal face of the locking pall on the distal face of the exterior wedge on the locking collar. Any proximally directed forces will merely urge the proximal face of the exterior wedge against the tapered shoulder of the needle holding member located adjacent the luer collar. Thus, the re-exposure of the used unit dose needle is positively prevented.

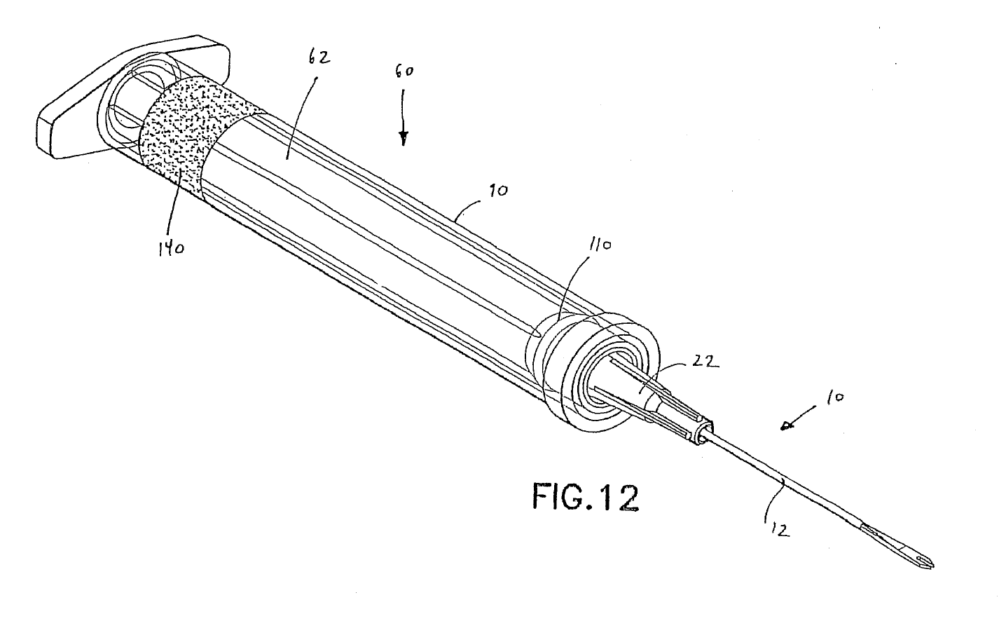

It is further contemplated that the shieldable medical assembly 8 may include means for indicating that the safety shield member 90 is in the shielding position, or more desirably, locked in place in the fully extended position shielding the unit dose needle. Such means may include a visual indicator, a tactile indicator, an audible indicator, or the like. For example, as shown in FIG. 12, corresponding visual structure may be present on the safety shield member 90 an the locking collar 110 to confirm proper engagement therebetween. In particular, safety shield member 90 may include a circumferential color band such as green band 140 at the proximal end 92 thereof. Locking collar 110 may further include a color band or may be constructed of a material which is colored in a complementary color of the color band on the safety shield member 90, such as red color. In this manner, when safety shield member 90 is extended to the shielding position, the green band 140 will overlap the red locking collar 110, thereby creating a black shaded color portion. This change in color provides a visual indication that the shieldable medical assembly 8 is in the shielding position and can be appropriately discarded. Also, the interaction between the locking shoulder 98 and the corresponding surfaces of the locking collar 110 create a tactile and or audible indication that the safety shield member 90 has been moved to the shielding position and locked in place.

The present invention provides an effective method for use of a unit dose medical needle by providing a unit dose needle with a proper handle portion and an appropriate shielding mechanism. In particular, the needle holding member 62 provides an effective handle portion for using the bifurcated needle 12 to administer a vaccine. Also, the overall dimensions of the safety shield member 90 provide an effective shield portion for the entire length of bifurcated needle 12 after use thereof.

It is important to note that as the unit dose needle is merely intended to deliver a unit dose of a vaccine which is present on the tip thereof, it is of solid construction. Accordingly, there is need for any internal fluid communication between the unit dose needle assembly 10 and the needle holding assembly 60. The needle holding assembly 60 is merely provided to act as a structure which is capable of providing an effective handle for the unit dose needle, and to provide for effective safety shielding of the unit dose needle after use thereof.

While the present application is satisfied by embodiments in may different forms, there is shown in the drawings and described herein in detail, the preferred embodiments of the invention, with the understanding that the present disclosure is to be considered as exemplary of the principles of the invention and is not intended to limit the invention to the embodiments illustrated. Various other embodiments will be apparent to and readily made by those skilled in the art without departing from the scope and spirit of the invention. The scope of the invention will be measured by the appended claims and their equivalents.

What is claimed is:

1. A shieldable unit dose assembly, comprising:
a needle holding member having a proximal end and a distal end and a cylindrical body extending between the proximal end and the distal end, wherein at least a portion of the cylindrical body is a solid member, the distal end including a male tapering surface;
a unit dose needle assembly supported at the distal end of the needle holding member, the unit dose needle assembly comprising a solid elongated unit dose needle having a patient end comprising a prong for containing a unit dose of a vaccine and for administering the unit dose directly from the prong to a patient, and a hub supporting the unit dose needle, the hub including a female tapering surface in engagement with the male tapering surface at the distal end of the needle holding member; and
a telescoping safety shield member disposed around the needle holding member and axially movable from a non-shielding position surrounding at least a portion of the needle holding member to a telescoped shielding position encompassing the patient end of the unit dose needle, and wherein the solid member of the cylindrical body and the solid unit dose needle cooperate to prevent fluid communication between the prong and the needle holding member.

2. The shieldable unit dose assembly of claim 1, wherein the needle holding member includes structure located proximate to the distal end adapted to restrain the telescoping safety shield in shielded position and prevent the safety shield member from returning to the non-shielding position.

3. The shieldable unit dose assembly of claim 2, wherein the structure of the needle holding member includes an external groove proximate the distal end with a locking member disposed around the needle holding member and including a portion cooperating with the external groove, and wherein the safety shield member includes an internal locking recess at a proximal end thereof adapted to cooperate with the locking member when the safety shield member is in the shielding position to prevent the safety shield member from returning to the non-shielding position.

4. The shieldable unit dose assembly of claim 3, wherein the locking member comprises a locking collar having an exterior wedge for engaging the internal locking recess to prevent the safety shield member from returning to the proximal position.

5. The shieldable unit dose assembly of claim 3, wherein the locking member comprises a locking collar having an interior wedge for engaging the external groove defined in the needle holding member.

6. The shieldable unit dose assembly of claim 3, wherein the locking member is tubular shaped and made of a resiliently deformable material such that the locking member may be positioned over the distal end of the needle holding member and resiliently engage the external groove defined in the needle holding member.

7. The shieldable unit dose assembly of claim 1, wherein the unit dose needle comprises a bifurcated needle having two pointed prongs capable of penetrating or abrading the skin of a patient, the prongs separated by a U-shaped channel capable of holding a unit dose of vaccine.

8. The shieldable unit dose assembly of claim 1, further comprising means for indicating that the safety shield member is in the shielding position.

9. The shieldable unit dose assembly of claim 1, wherein the distal end of the needle holding member includes an annular collar adjacent the male tapering surface including internal threads, and wherein the hub includes structure for threaded engagement with the internal threads of the annular collar when the female tapering surface is in engagement with the male tapering surface.

10. The shieldable unit dose assembly of claim 1, further comprising structure for maintaining the safety shield member in the non-shielding position prior to movement to the shielded position.

11. The shieldable unit dose assembly of claim 1, wherein the safety shield member includes a plurality of internal ribs extending axially along an internal surface thereof.

12. The shieldable unit dose assembly of claim 1, further comprising a removable cover enclosing the unit dose needle and frictionally engaging the hub.

13. The shieldable unit dose assembly of claim 1 wherein the distal end of the needle holding member comprises a solid member and wherein the solid member of the distal end, the solid member of the cylindrical body and the solid unit dose needle cooperate to prevent fluid communication between the bifurcated tip and the needle holding member.

14. A shieldable unit dose assembly, comprising:
a needle holding member having a proximal end and a distal end wherein at least a portion of the distal end comprises a solid member, the distal end including a male tapering surface;
a unit dose needle assembly supported at the distal end of the needle holding member, the unit dose needle assembly comprising a solid elongated unit dose needle having a patient end comprising a prong for containing a unit dose of a vaccine and for administering the unit dose directly from the prong to a patient, and a hub supporting the unit dose needle, the hub including a female tapering surface in engagement with the male tapering surface at the distal end of the needle holding member; and
a telescoping safety shield member disposed around the needle holding member and axially movable from a non-shielding position surrounding at least a portion of the needle holding member to a telescoped shielding position encompassing the patient end of the unit dose needle, and wherein the solid member of the distal end and the solid unit dose needle cooperate to prevent fluid communication between the prong and the needle holding member.

15. A shieldable unit dose assembly, comprising:
a needle holding member having a proximal end and a distal end and a cylindrical body extending between the proximal end and the distal end, wherein at least a portion of the cylindrical body is a solid member, the needle holding member defining an external groove proximate the distal end, the distal end including a male tapering surface and an annular collar including internal threads adjacent the male tapering surface;
a locking member disposed around the needle holding member and including a portion cooperating with the external groove;
a unit dose needle assembly supported at the distal end of the needle holding member, the unit dose needle assembly comprising a solid elongated unit dose needle having a patient end including two pointed prongs capable of penetrating or abrading the skin of a patient and separated by a U-shaped channel capable of holding a unit dose of vaccine, wherein the solid member of the cylindrical body and the solid unit dose needle cooperate to prevent fluid communication between the two pointed prongs and the needle holding member, and a hub supporting the unit dose needle, the hub including a female tapering surface in engagement with the male tapering surface at the distal end of the needle holding member and including structure for threadably engaging the internal threads of the annular collar; and
a telescoping safety shield member disposed around a portion of the needle holding member and locking member and axially movable from a non-shielding position encircling the needle holding member to a telescoped shielding position encompassing the patient end of the unit dose needle, the safety shield member defining an internal locking recess at a proximal end thereof adapted to cooperate with the locking member in the shielding position of the safety shield member to restrain the telescoping safety shield in shielded position and prevent the safety shield member from returning to the non-shielding position.

16. The shieldable unit dose assembly of claim 15, wherein the locking member comprises a locking collar having an exterior wedge for engaging the internal locking recess to prevent the safety shield member from returning to the non-shielding position.

17. The shieldable unit dose assembly of claim 15 wherein the locking member is tubular shaped and made of a resiliently deformable material such that the locking member may be positioned over the distal end of the needle holding member and resiliently engage the external groove defined in the needle holding member.

18. The shieldable unit dose assembly of claim 15, further comprising a removable safety cap enclosing the unit dose needle and frictionally engaging the hub.

19. A shieldable unit dose assembly, comprising:
a solid unit dose needle assembly comprising a hub having a female tapering surface at a proximal end thereof and a solid elongated unit dose needle extending from a distal end thereof, the unit dose needle having a length capable of retrieving a unit dose of a vaccine from a separate container and having a patient end comprising a prong for containing and administering the unit dose of a vaccine directly from the prong to a patient; and
a needle holding assembly having an elongated body with a proximal end and a distal end, at least a portion of the elongated body and/or the distal end comprising a solid member, the distal end including a male tapering surface in engagement with the female tapering surface of the hub of the unit dose needle assembly and an annular collar having internal threads in threaded engagement with corresponding structure on the proximal end of the hub, wherein the solid member of the elongated body and/or the distal end cooperates with the solid unit dose needle to prevent fluid communication between the prong and the needle holding assembly the needle holding assembly further including a telescoping safety shield member disposed concentrically around the elongated body with at least a portion of the proximal end of the body extending from the safety shield member, the safety shield member being axially movable from a non-shielding position in which the unit dose needle is exposed to a telescoped shielding position encompassing the length of the unit dose needle including the patient end.

20. The shieldable unit dose assembly of claim 19, wherein the elongated body includes an external groove proximate the distal end thereof, and the needle holding assembly further comprises a locking collar disposed about a portion of the elongated body cooperating with the external groove, and wherein the safety shield member includes an internal locking recess at a proximal end thereof adapted to cooperate with the locking collar when the safety shield member is in the shielding position to prevent the safety shield member from returning to the non-shielding position.

21. The shieldable unit dose assembly of claim 20, wherein the unit dose needle comprises a bifurcated needle having two pointed prongs capable of penetrating or abrading the skin of a patient, the prongs separated by a U-shaped channel capable of holding a unit dose of vaccine.

22. The shieldable unit dose assembly of claim 19, further comprising a removable safety cap enclosing the unit dose needle and frictionally engaging the hub.

23. A method for administering a vaccine comprising:
providing a shieldable medical needle assembly comprising a unit dose needle assembly attached to a needle holding assembly, the unit dose needle assembly including a solid elongated unit dose needle including a bifurcated pronged patient end for containing a unit dose of a vaccine, the needle holding assembly having an elongated body with a proximal end and a distal end, at least a portion of the elongated body and/or the distal end comprising a solid member, wherein the solid member of the elongated body and/or the distal end cooperates with the solid unit dose needle to prevent fluid communication between the bifurcated pronged patient end and the needle holding assembly, the needle assembly further including a telescoping safety shield member disposed concentrically around the elongated body with at least a portion of the proximal end of the body extending from the safety shield member;
contacting the bifurcated pronged patient end of the unit dose needle with a vaccine so as to contain at least a portion of the vaccine on the bifurcated pronged patient end;
contacting the bifurcated pronged patient end of the unit dose needle with a patient's skin so as to cause the bifurcated pronged patient end to abrade the patient's skin; and
axially moving the safety shield member in a direction toward the bifurcated pronged patient end from a non-shielding position in which the safety shield member is disposed concentrically around the elongated body and the unit dose needle is exposed to a telescoped shielding position encompassing the length of the unit dose needle including the bifurcated pronged patient end, wherein said needle holding member includes structure located proximate the distal end adapted to restrain the shield in the shielded position.
